(19) United States
(12) Patent Application Publication
(10) Pub. No.: US 2004/0097596 A1
(43) Pub. Date: May 20, 2004

(54) Method for combating attack and spread of fungal pathogens in plants

(76) Inventors: Maarten Hendrik Stuiver, Neustadt an der Weinstrasse (DE); Naomi Pain, Berkshire (GB)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/239,700

(22) PCT Filed: Mar. 12, 2001
(86) PCT No.: PCT/EP01/02725

(30) Foreign Application Priority Data
Mar. 15, 2000 (GB) 0006244.8

(51) Int. Cl. A01H 1/00; C12N 15/82; A01N 37/18

(52) U.S. Cl. 514/617; 800/279

(57) ABSTRACT

The present invention concerns a method for introducing and/or improving plant resistance to the attack and/or spread of fungal pathogens. The method comprises applying at least one anti-fungal compound to a plant or plant part that has been genetically modified using recombinant DNA technology. The method results in a synergistic effect, as determined using Colby's formula, between the anti-fungal compound and the genetically modified plant or plant part which leads to improve resistance relative to control plants.

This is a § 371 of PCT/EP01/02725, filed Mar. 12, 2001, and published Sep. 20, 2001, as WO 01/67865, which claims priority of Great Britain Patent Application No. 0006244.8 filed Mar. 15, 2000.

The present invention concerns a method for combating the attack and spread of fungal pathogens in plants, which method comprises combining chemical and genetic means to control fungal pathogens.

Although many fungi are non-pathogenic, some pathogenic fungi are responsible for great annual economic loss in commercially important crops. It has been reported that in rice and wheat 15% of the yield is lost mainly because of diseases caused by fungi, while losses in potato yield due to infection with Phytophthora infestans amount from 10% to 25% (Oerke, E.C. et al. Crop production and crop protection. Elsevier, Amsterdam, 1994).

Various chemical and genetic approaches to combating fungal pathogens are known.

Examples of chemical approaches to combating the attack and/or spread (in plants) of various fungal pathogens include treating the plant with any of the following: for example, aromatic hydrocarbons and derivatives thereof, such as hexachlorobenzene, pentachloronitrobenzene (quintozene), tetrachloronitrobenzene (tecnazene), biphenyl and o-phenylphenol; chlorothalonil; dicloran; etridiazole; dicarboximides, such as procymidone, iprodione, vinclozolin and chlozolinate; carboxamides, such as carboxin and oxycarboxin; morpholines, such as dodemorph, tridemorph, aldimorph and fenpropimorph; phenylpyrroles, such as fenpiclonil; piperidines, such as fenpropidin; azoles, including imidazoles, such as imazalil, prochloraz, triflumizole and triazoles, such as triadimefon, triadimenol, bitertanol, cyproconazole, propiconazole, epoxiconazole, penconazole, flutriafol, flusilazole, diniconazole, myclobutanil; benzimidazoles, such as benomyl and carbondazim; phenylamides, such as metalaxyl, furalaxyl, benalaxyl, ofurace and oxadixyl; 2-aminopyimidines, such as dimethirimol, ethirimol and bupirimate; organophosphorous compounds, such as pyrazophos, tolclofos-methyl and edifenphos; and strobilurins, including β-methoxyacrylates, such as azoxystrobin and picoxystrobin, methoxyiminoacetates, such as kresoxim-methyl and trifloxystrobin and methoxyiminoacetamides, such as metominostrobin.

Various genetic approaches to combating fungal pathogens have also been described. Resistance to various fungi (or to specific fungi) may be introduced to the plant or existing resistance levels may be improved or enhanced. For example, such introduction or enhancement of resistance to fungi may be achieved by expressing, in a plant, an antifungal agent, such as an antifungal protein, a phytoalexin or a saponin and/or an agent able to trigger a hypersensitive response in the plant.

However, there are several diseases caused by fungi for which, at present, no suitable chemical treatment exists (for example, there is currently no effective chemical treatment against Sclerotinia which is responsible for great annual losses in sunflower, sugar beet and canola). Similarly, there are several diseases caused by fungi for which no effective genetic control means has yet been developed. Known chemical and genetic approaches to combating the attack and spread of fungi in plants are often only effective against a narrow spectrum of fungal pathogens and may not provide the plant with resistance over an extended period of time. Other disadvantages associated with known anti-fungal approaches include, in some cases, the local applicability and the length of time taken for the anti-fungal means to become effective and to start combating infection.

A method combining chemical and genetic approaches to combating fungal pathogens is proposed in UK patent application GB2333043 which discusses use of a fungicide on a plant into which has been introduced, by genetic modification, a trait conferring fungal resistance. Although a beneficial effect is described, this document does not exemplify any effect at all, and certainly does not show synergistic effects. Published international patent application WO 98/17115 discusses a method for controlling parasitic fungi in cultivated plants, which plants have modified pathogenic resistance against certain parasitic fungi, and which method involves treating the cultivated plants with an active substance from the strobilurin class. The above documents, mentioning the possibility of a combined chemical and genetic approach, do not exemplify synergy. International patent application WO 98/29537 shows the effects of fungicides on so-called ‘immunomodulated’ plants (plants that have either been chemically treated or genetically modified such that induction of part of the plants defence compounds takes place (often referred to as ‘salicylic acid-dependent defence’), or such that the plants are more readily able to induce that part of their defence). The induced defence compounds appear to represent part of the total defence potential of the plant. It is apparent that there is synergy between this part of the plant's endogenous defence and fungicides. However, the limitation of this technology is that stimulation of the salicylic acid-dependent defence has effect on some, but not on other fungi (Thomma et al. Current Opinion Immunology 2001, 13(1), 63-68). Since most crop plants can be infected by different fungi, the use of this invention is limited.

Despite the various available means for combating fungal pathogens, there remains a need for improved anti-fungal treatments. The present invention alleviates some of the problems associated with conventional anti-fungal approaches and provides effective means to resist the attack and combat the spread, in plants, of a broad range of pathogenic fungi. The present invention offers a method of introducing or improving/enhancing resistance in plants to various fungal pathogens and enables a reduced quantity of fungicide to be used on plants/crops whilst retaining disease occurrence at the same or reduced level.

We have found that by combining chemical and genetic means to control fungal pathogens, a synergistic effect is achieved enabling the attack and spread of fungal pathogens in plants to be effectively combated. More specifically, we have found that application of anti-fungal compounds/fungicides, to genetically modified plants (genetically modified to express at least one agent able to trigger a hypersensitive response in a plant) results in a synergistic effect, thereby conferring increased fungal resistance to the plant relative to plants treated according to a non-combined approach.

According to the present invention, there is provided a method for introducing and/or improving, in a plant, resistance to the attack and/or spread of fungal pathogens, which method comprises applying at least one anti-fungal compound/fungicide to a plant or plant part which has been genetically modified to express at least one agent able to trigger a hypersensitive response in a plant, wherein said agent synergistically enhances said plant resistance.

According to a further embodiment of the present invention, there is provided a plant having improved resistance to fungal pathogens, said plant having been genetically modified to express at least one agent able to trigger a hypersensitive response in a plant, wherein said plant has been treated with at least one anti-fungal compound/fungicide. Alternatively, the plant may be treated at least once with an anti-fungal compound prior to genetic modification and optionally also treated at least once with an anti-fungal agent after genetic modification. The plant having improved resistance to fungal pathogens (obtained by the method according to the present invention) may be used as a parent in conventional plant breeding crosses to develop hybrids and lines having improved fungal resistance.

Advantageously, the combined approach defined in the method according to the present invention enables achievement of a synergistic effect, as determined using Colby's Formula (Colby, S. R. Weeds 1967, 15, 20-22—Calculating Synergistic and Antagonistic Responses of Herbicide Combinations). A synergistic effect according to the present invention applies in cases where the value for expected disease control is lower than the observed value for disease control. The formula below, which is based on Colby's Formula, was used to determine whether a synergistic effect was achieved.

Formula used to Determine Synergy

E=(100 —% disease control for untreated genetically modified plant alone)×(% disease control for relevant chemical & rate alone)/100+% disease control for untreated genetically modified plant alone

E=Expected % disease control

The occurrence of fungal disease in plants, treated according to the method of the invention, is reduced relative to control plants (genetically modified plants not treated with fungicide or non-genetically modified plants treated with fungicide).

Reference to the term “plant” herein comprises both whole plants (including seedlings, bushes and trees) and plant parts (including seed) having modified resistance to fungal pathogens. Advantageously, the method according to the present invention is applicable to angiosperms and gymnosperms, monocotyledonae and dicotyledonae. According to a preferred feature of the present invention, the plant is a field crop, such as potato, banana, coffee, rape seed, turnip, asparagus, tea, tomato, onion, rice, wheat, barley, oats, maize, canola, sunflower, tobacco, sugar beet, cotton, soya, sorghum, mangoes, peaches, apples, pears, strawberries, melons, carrot, lettuce and cabbage. Further preferably, the plant is potato, tomato, banana, tobacco, canola, sunflower or wheat.

Fungal pathogens which may be controlled by the method according to the present invention, are selected from fungal pathogens falling within the following phyla: Acrasiomycota, Ascomycota, Basidiomycota (Basidiomycetes, Teliomycetes, Ustomycetes) Chytridiomycota, Dictyosteliomycota, Hyphochytriomycota, Labyrinthulomycota, Myxomycota, Oomycota, Plasmodiophoromycota, and Zygomycota (Trichomycetes, Zygomycetes). More specifically, the combined approach defined by the present invention may be used to control one or more of the following pathogens: Pyricularia oryzae (Magnaporthes grisea) on rice and wheat and other Pyricularia spp. on other hosts; Puccinia recondita, Puccinia striiformis, Puccinnia graminis tritici and other rusts on wheat, Puccinia hordei, Pucciinia striifomis and other rusts on barley, and rusts on other hosts (for example turf, lye, coffee, pears, apples, peanuts, sugar beet, vegetables and ornamental plants); Erysiphe cichoracearum on cucurbits (for example melon); Erysiphe graminis (powdery mildew) on barley, wheat, rye and turf and other powdery mildews on various hosts, such as Sphaerotheca macularis on hops, Sphaerotheca fusca (Sphaerotheca fuliginea) on cucurbits (for example cucumber), Leveillula taurica on tomatoes, aubergine and green pepper, Podosphaera leucotricha on apples and Uncinula necator on vines; Cochliobolus spp., Helminthosporium spp., Drechslera spp. (Pyrenophora spp.), Rhynchosporium spp., Mycosphaerella graminicola (Septoria tritici) and Phaeosphaeria nodorum), (Stagonospora nodorum or Septoria nodorum), Pseudocercosporella herpotrichoides and Gaeumannomyces graminis on cereals (for example wheat, barley, rye), turf and other hosts; Cercospora arachidicola and Cercosporidium personatum on peanuts and other Cercospora spp. on other hosts, for example sugar beet, bananas, soya beans and rice; Botrytis cinerea (grey mould) on tomatoes, strawberries, vegetables, vines and other hosts and other Botrytis spp. on other hosts; Alternaria spp. on vegetables (for example carrots), oil-seed rape, apples, tomatoes, potatoes, cereals (for example wheat) and other hosts; Venturia spp. (including Venturia inaequalis (scab)) on apples, pears, stone fruit, tree nuts and other hosts; Cladosporium spp. on a range of hosts including cereals (for example wheat) and tomatoes; Monilinia spp. on stone fruit, tree nuts and other hosts; Didymella spp. on tomatoes, turf, wheat, cucurbits and other hosts; Phoma spp. on oil-seed rape, turf, rice, potatoes, wheat and other hosts; Aspergillus spp. and Aureobasidium spp. on wheat, lumber and other hosts; Ascochyta spp. on peas, wheat, barley and other hosts; Stemphylium spp. (Pleospora spp.) on apples, pears, onions and other hosts; summer diseases (for example bitter rot (Glomerella cingulata), black rot or frogeye leaf spot (Botryosphaeria obtusa), Brooks fruit spot (Mycosphaerella pomi), Cedar apple rust (Gymnosporangium juniperi-virginianae), sooty blotch (Gloeodes pomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. on soybeans, tobacco, onions and other hosts, Pseudoperonospora humuli on hops and Pseudoperonospora cubensis on cucurbits; Pythium spp. (including Pythium ultimum) on turf and other hosts; Phytophthora infestans on potatoes and tomatoes and other Phytophthora spp. on vegetables, strawberries, avocado, pepper, ornamentals, tobacco, cocoa and other hosts; Thanatephorus cucumeris on rice and turf and other Rhizoctonia spp. on various hosts such as wheat and barley, peanuts, vegetables, cotton and turf; Sclerotinia spp. on turf, peanuts, potatoes, oil-seed rape and other hosts; Sclerotium spp. on turf, peanuts and other hosts; Gibberella fujikuroi on rice; Colletotrichum spp. on a range of hosts including turf, coffee and vegetables; Laetisaria fucifomis on turf; Mycosphaerella spp. on bananas, peanuts, citrus, pecans, papaya and other hosts; Diaporthe spp. on citrus, soybean, melon, pears, lupin and other hosts; Elsinoe spp. on citrus, vines, olives, pecans, roses and other hosts; Verticillium spp. on a range of hosts including hops, potatoes and tomatoes; Pyrenopeziza spp. on oil-seed rape and other hosts; Oncobasidium theobromae on cocoa causing vascular streak dieback; Fusarium. spp., Typhula spp., Microdochium nivale, Ustilago spp., Urocystis spp., Tilletia spp. and Claviceps purpurea on a variety of hosts but particularly wheat, barley, turf and maize; Ramularia spp. on sugar beet, barley and other hosts; post-harvest diseases particularly of fruit (for example Penicillium digitatum, Penicillium italicum and Trichoderma viride on oranges, Colletotrichum musae and Gloeosporium musarum on bananas and Botrytis cinerea on grapes); other pathogens on vines, notably Eutypa lata, Guignardia bidwellii, Phellinus igniarus, Phomopsis viticola, Pseudopeziza tracheiphila and Stereum hirsutum; other pathogens on trees (for example Lophodermium seditiosum) or lumber, notably Cephaloascus fragrans, Ceratocystis spp., Ophiostoma piceae, Penicillium spp., Trichoderma pseudokoningii, Trichoderma viride, Trichoderma harzianum, Aspergillus niger, Leptographium lindbergi and Aureobasidium pullulans; and fungal vectors of viral diseases (for example Polymyxa graminis on cereals as the vector of barley yellow mosaic virus (BYMV) and Polymyxa betae on sugar beet as the vector of rhizomania). According to a preferred feature of the present invention, fungal pathogens which may be controlled by the method according to the present invention are selected from Magnaporthe grisea on rice; Erysiphe graminis on wheat; Septoria tritici on cereals; Botrytis cinerea on tomatoes and vines; Cladosporium spp. on tomatoes; Oidium lycopernicon on tomatoes; Phoma spp. on oil-seed rape; Phytophthora infestans on potatoes and tomatoes; Sclerotinia spp. on oil-seed rape and sunflower; Peronospora tabacina on tobacco; Stagonospora nodorum on wheat; and Mycosphaerella spp. on bananas.

The aforementioned fungal pathogens are responsible for diseases that cause great annual loss in commercially important crops. Therefore, the improved resistance to fungal attack and spread (resulting from the combined approach defined by the present invention) is likely to be of great economic and environmental value. A further advantage of the present invention is the increase in the range of fungi that can be combated using the combined approach defined by the present invention.

According to a preferred feature of the present invention, the anti-fungal compound/fungicide is selected from one or more, or a combination, of any of the following: azoxystrobin (Amistar™, Abound™, Heritage™, Quadris™); chlorothalonil (Bravo™, Daconil™, Tattoo™, Dacostar™, Vanox™); hexaconazole (Anvil™, Planete™); flutriafol (Impact™, Ferrax™, Vincit™; oxadixyl, cymoxanil, mancozeb (Trustan™); fluazinam (Shirlan™); bupirimate (Nimrod™); diethofencarb (Sumico™, Getter™, Sumiblend™); dimethirimol (Milcurb™); ethirimol (Milcurb™, Milgo™, Milstem™, Halley™); procymidone; metalaxyl (Ridomil™, Ridomil Gold™, Fubol™, Osprey™); or any other fungicide. Further preferably, the anti-fungal compound comprises at least chlorothalonil, azoxystrobin or metalaxyl. The anti-fungal compound may be a protective compound, i.e. a compound that is typically used for prophylactic purposes and which does not substantially penetrate into the plant; or the compound may be a curative compound, i.e. a compound that is typically used following disease occurrence and which compound is systemically acting (able to establish itself within the plant tissue); or the anti-fungal compound may be a combination of one or more protective and curative compounds.

According to a preferred feature of the present invention, the anti-fungal compound is administered to the plant or plant part in the form of a composition.

In order to apply the anti-fungal compound to a plant, to a seed of a plant, to the locus of the plant or seed, to soil or to any other growth medium, the anti-fungal compounds usually formulated into a composition which includes, in addition to the anti-fungal compound, a suitable inert diluent or carrier and, optionally, a surface active agent (SFA). SFAs are chemicals able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of an anti-fungal compound. The composition is generally used for the control of fungi such that the anti-fungal compound is applied at a rate of from 0.1 kg to 10 kg per hectare, preferably from 1 g to 6 kg per hectare, more preferably from 1 g to 1 kg per hectare.

When used in a seed dressing, the anti-fungal compound is used at a rate of 0.0001 g to 10 g (for example 0.001 g or 0.05g), preferably 0.005g to 10 g, more preferably 0.005 g to 4 g, per kilogram of seed.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the anti-fungal compound.

Dustable powders (DP) may be prepared by mixing the anti-fungal compound with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing the anti-fungal compound with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing the anti-fungal compound with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of the anti-fungal compound and one or more powdered solid diluents or carriers, or from pre-formed blank granules by absorbing the anti-fungal compound (or a solution thereof, in a suitable agent) in a porous granular material (such as pumice, attapulgite clays, fuller's earth, kieselguhr, diatomaceous earths or ground corn cobs) or by adsorbing the anti-fungal compound (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticlding agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving the anti-fungal compound in water or an organic solvent, such as a ketone, alcohol or glycol ether. These solutions may contain a surface-active agent (for example to improve water dilution or prevent crystallisation in a spray tank).

Emulsifiable concentrates (EC) or oil-in-water emulsions (EW) may be prepared by dissolving the anti-fungal compound in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves providing the anti-fungal compound either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. The anti-fungal compound is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of the anti-fungal compound. SCs may be prepared by ball or bead milling the solid anti-fungal compound in a suitable medium, optionally with one or more dispersing agents, to produce a fine particle suspension of the anti-fungal compound. One or more wetting agents may be included in the composition and a suspending agent may be included to reduce the rate at which the particles settle. Alternatively, the anti-fungal compound may be dry milled and added to water, containing agents hereinbefore described, to produce the desired end product.

Aerosol formulations comprise the anti-fungal compound and a suitable propellant (for example n-butane). The anti-fungal compound may also be dissolved or dispersed in a suitable medium (for example water or a water miscible liquid, such as n-propanol) to provide compositions for use in non-pressurised, hand-actuated spray pumps.

The anti-fungal compound may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains the anti-fungal compound and, optionally, a carrier or diluent therefor. The polymeric shell may be produced by either an interfacial polycondensation reaction or by a coacervation procedure. The compositions may provide for controlled release of the anti-fungal compound and they may be used for seed treatment. The anti-fungal compound may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of the anti-fungal compound). Such additives include surface-active agents, spray additives based on oils, for example certain mineral oils or natural plant oils (such as soy bean and rape seed oil), and blends of these with other bio-enhancing adjuvants (ingredients which may aid or modify the action of the anti-fungal compound).

The anti-fungal compound may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SPAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The anti-fungal compound may be applied by any of the known means of applying fungicidal compounds. For example, it may be applied, formulated or unformulated, to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

The anti-fungal compound may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of the anti-fungal compound (for example 0.0001 to 10%, by weight) depending upon the purpose for which they are to be used.

The compositions for use in this invention may contain other compounds having biological activity, for example micronutrients or compounds having similar or complementary fungicidal activity or which possess plant growth regulating, herbicidal, insecticidal, nematicidal or acaricidal activity.

By including another fungicide, the resulting composition may have a broader spectrum of activity or a greater level of intrinsic activity than by use of one anti-fungal compound alone. Furthermore, the additional fungicide(s) may have an additional synergistic effect on the synergistic fungicidal activity of the first anti-fungal compound in combination with the genetically modified plants.

The first anti-fungal compound may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the first anti-fungal compound; or help to overcome or prevent the development of resistance to individual components. The particular additional active ingredient will depend upon the intended utility of the composition.

Examples of fungicidal compounds which may be included in the composition for use in the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethylbenzimidazole-1-sulphonamide, α-[N-(3-chloro-2,6-xylyl)-2-methoxyacetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro- 1-ethyl-1-methyl-2-oxopropyl)4-methylbenzamide (RH-7281, zoxamide), N-allyl4,5,-dimethyl-2-trimethylsilylthiophene-3-carboxamide (MON65500), N-(1-cyano- 1,2-dimethylpropyl)-2-(2,4-dichlorophenoxy) propionamide (AC382042), N-(2-methoxy-5-pyridyl)-cyclopropane carboxamide, acibenzolar (CGA245704), alanycarb, aldimorph, ancymidal, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chloroneb, chlorothalonil, chlorozolinate, clozylacon, copper containing compounds such as copper hydroxide, copper oxychloride, copper oxyquinolate, copper sulphate, copper tallate and Bordeaux mixture, cymoxanil, cyproconazole, cyprodinil, debacarb, di-2-pyridyl disulphide 1,1′-dioxide, dichlofluanid, dichlone, dichlozoline, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl(Z)-N-benzyl-N([methyl(methyl-thioethylideneaminooxycarbonyl) amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferimzone, fluazinam, fludioxonil, flumetover, flumorph (SYP-LI90), fluoroimide, fluquinconazole, flusilazole, flusolfamide, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kcresoxim-methyl, LY186054, LY211795, LY248908, mancozeb, maneb, mefenoxam, mepanipyrim, mepronil, metalaxyl, R-metalaxyl (metalaxyl-M), metconazole, methasulfocarb, metiram, metiram-zinc, metominostrobin, myclobutanil, myclozoline, neoasozin, nickel dimethyldithiocarbamate, nitrothal-isopropyl, nuarimol, ofurace, organomercury compounds, oxadixyl, oxasulfuron, oxolinic acid, oxpoconazole, oxycarboxin, pefurazoate, penconazole, pencycuron, phenazin oxide, phosdiphen, phosetyl-Al, phosphorus acids, phthalide, picoxystrobin (ZA1963) polyoxin D, polyram, probenazole, prochloraz, procymidone, propamocarb, propiconazole, propineb, propionic acid, pyraclostrobin (BAS 500F), pyrazophos, pyrifenox, pyrimethanil, pyroquilon, pyroxyfur, pyrrolnitrin, quaternary ammonium compounds, quinomethionate, quinoxyfen, quintozene, simeconazole, sipconazole (F-155), sodium pentachlorophenate, spiroxamine, streptomycin, sulphur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The anti-fungal compounds may be mixed with soil, peat or other rooting media for the protection of plants against seed-borne, soil-borne or foliar fungal diseases.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

According to the present invention, a plant or plant part, to which the anti-fungal compound is applied, is genetically modified to introduce and/or enhance fungal resistance by expression of at least one agent able to trigger a hypersensitive response in the plant.

A hypersensitive response (HR) is a localised response to a pathogen (such as a fungal pathogen) and results in the rapid death of infected plant cells, thereby stopping spread of the infection. The HR is also associated with secondary responses, such as callus deposition, generation of active oxygen species, induction of phytoalexins, changes in ion fluxes across membranes and induction of acquired resistance (AR).

Examples of agents able to trigger an HR in plants include agents involved in the gene-for-gene resistance interaction. The gene-for-gene hypothesis proposes that interaction between pathogen and plant takes place via a specific receptor-ligand recognition system, the receptor being a plant-expressed protein and the ligand being a pathogen-expressed protein. Recognition, either directly or indirectly, of the pathogen-expressed (avirulence/elicitor) protein by the plant (resistance) protein triggers an HR in the plant. The avirulence-resistance protein interaction is highly specific with a given resistance gene only conferring resistance if a pathogen expresses a complementary avirulence gene. Examples of agents involved in the gene-for-gene resistance interaction include avirulence genes cloned from bacterial pathogens (such as Pseudomonas and Xanthomanas) and from fungal pathogens (such as Cladosporium fulvum, Rhynchosporium secalis and Phytophthora parasitica). Plant genes coding for some of the corresponding resistance genes have also been cloned (such as the tomato Cf9 gene corresponding to the avirulence gene Avr9 from Cladosporium fulvum, and the Rpm1 gene from Arabidopsis, corresponding to the avirulence gene AvrRpm1 from Pseudomonas).

According to a preferred feature of the present invention, the agent able to trigger an HR in a plant is a pathogen avirulence gene encoding a specific elicitor or a functional part thereof, which avirulence gene, and preferably a corresponding resistance gene, is introduced into a plant genome. Further preferably, the avirulence gene is Avr9 from Cladosporium fulvum and the resistance gene is Cf9 from tomato.

Advantageously, expression of avirulence and resistance genes can be regulated such that simultaneous expression of the genes only occurs at the site of infection and on induction by a pathogen. If the plant does not contain a corresponding resistance gene, such a gene can be introduced into the plant either by genetic modification or by conventional breeding techniques. The above means for introducing fungal resistance is discussed more fully in international patent application WO 91/15585, which is incorporated herein by reference.

Further means for genetically modifying plants, to introduce or improve plant resistance to fungi, include introduction into a plant of a plant signal transduction protein or a homologue thereof which, when expressed, gives rise to an HR in the plant.

Plant resistance proteins, when activated by interaction with pathogen-derived elicitor proteins, are capable of inducing a signal transduction pathway. Some interactions are believed (at least in part) to use a common pathway (Century, K.S., et al., Science 278, 1963-1965, 1997). Century et al. reported the NDR1 locus to be required for resistance to the bacterial pathogen Pseudomonas syringae pv. tomato and the fungal pathogen Peronospora parasitica. Sirnilarly, Parker, J.E., et al. (The Plant Cell 8, 2033-2046, 1996) demonstrated the product encoded by the eds1-locus in Arabidopsis thaliana to have a key function in the signal transduction pathway following infection with Peronospora parasitica, but not after infection with Pseudomonas syringae pv glycinae. It has been further reported that plant-derived proteins can also elicit cell-death like phenomena (Karrer, E.E. et al., Plant Mol. Biol. 36, 681-690, 1998). Karrer et al. reported 11 clones able to produce lesions in tobacco plants.

There are several ways to trigger an HR in plants using a plant signal transduction protein or a homologue or mutant thereof. Some of these methods are discussed below.

(i) Over-expression of Proteins Involved in Hypersensitive Lesion Formation.

Various methods for over-expression of proteins are well known within the art. The protein to be over-expressed may be a receptor of a ligand that normally triggers an HR, or a positive acting component in the signal transduction pathway leading to an HR. Over-expression of a positive regulator of the pathway (such as G-protein, kinase or phosphatase) can upset the balance between components of the signalling pathway, in turn leading to an HR. Therefore, inadvertent signalling, in the absence of the ligand normally responsible for triggering of the pathway, takes place.

(ii) Down Regulation, Inhibition or Inactivation of Negative Acting Components in the Signal Transduction Pathway Leading to an HR.

Various methods for the under-expression or down regulation of proteins are also well known within the art. The negative-acting proteins involved in the signal transduction pathway may have a variety of functions. Well known examples include phosphatases and kinases (common regulators of enzyme and signal transduction component activity). Pathogen-induced removal of such a protein can effectively be mediated through induced expression of antisense RNA (Kumlia et al., 1998, Current Science 74, 35-41); short stretches of sense RNA (van Bloldand et al., 1994 Plant Journal 6, 861-877); the expression of ribozymes, sequence specific RNA-based ribonucleases (see, for example, Wegener et al., 1994, Mol. Gen. Genet. 245, 465-470; Peiriman et al., 1995, Proc. Natl. Acad. Sci. USA 92, 6175-6179); or through RNAi (Fire et al., 1998, Nature 391, 806-811). A further possibility is expression in plants of proteins (such as antibodies) able to interfere with the normal inhibitory function of the negative-acting proteins thereby alleviating their inhibitory effect.

(iii) Dominant Interfering Proteins.

It is known that mutant proteins (such as point mutants and deletion mutants) derived from proteins with a role in signal transduction, can have altered properties. For example, the activity of mutant proteins can continuously be expressed in an active form, whereas the activity of the non-mutated counterpart is tightly regulated (Chang & Meyerowitz, 1995, Proc. Natl. Acad. Sci. USA 92, 4129-4133; Miloso et al., 1995, J. Biol. Chem. 270, 19557-19562). When such a mutant protein can be identified/constructed from one having a positively acting role in the signal transduction pathway leading to the hypersensitive reaction, it then can be used as a tool to obtain broad-spectrum resistance. By coupling the open reading frame encoding such active mutant protein to a pathogen-inducible promoter in a functional manner, activation of the signal transduction pathway leading to the HR is directly mediated through promoter activation by pathogen infection. Similarly, dominant interfering negative acting proteins are described (Boylan et al., 1994, Plant Cell 6, 449-460; Okamoto et al., 1997 Plant Physiol. 115,79-85; McNellis et al., 1996, Plant Cell 8, 1491-1503; Emmler et al., 1995, Planta 197, 103-110).

(iv) 2nd Messenger Generating Systems

Another way to induce the signal transduction pathway leading to an HR is through second messengers. Signal transduction, leading to an HR, is known to be mediated by second messenger molecules. For example, influx of Ca²⁺ ions appears to play an important role (Cho, Abstract ISPMB congress Singapore, 1997). It is possible to generate such a stimulus by introduction of a heterologous protein that allows unregulated Ca²⁺ influx into the cytoplasm, setting off the downstream sequence of events that eventually lead to an HR.

Therefore, according to a further preferred feature of the present invention, the agent able to trigger an HR in a plant is a plant signal transduction protein or a homologue or mutant thereof.

According to a preferred feature of the present invention, the signal transduction protein is ndr1 or a homologue thereof, eds1 or a homologue thereof, or Xa21 or a homologue thereof. The signal transduction protein may also be selected from G-proteins, a protein kinase and/or a protein phosphatase. The signal transduction protein may also be a mutant which, when expressed, is able to give rise to an HR in the plant. Preferred mutants include ndr1-CDPK and truncated Xa21 (as discussed in international application WO 99/45129, incorporated herein by reference).

Further means for conferring fungal resistance in plants include expression in a plant of an agent able to alleviate the inhibitory effect of a protein in the signal transduction pathway responsible for HR in plants. Examples of such agents include mRNA coding for an inhibitory protein in an anti-sense orientation; agents able to sterically interact with the inhibitory protein; an antibody; a ribozyme; or a single-stranded or double-stranded RNA molecule able to suppress translation of the mRNA coding for the inhibitory protein. Again, the above means for conferring fungal resistance is discussed in international application WO 99/45129.

In the case where expression of an anti-fungal agent results in an HR in the plant, it is important that the anti-fungal agent is placed under the control of a pathogen-inducible promoter. Examples of pathogen inducible promoters include the prp1 promoter (Martini, N., et al., Mol. Gen. Genet. 236, 179-186, 1993), Fis1 promoter (WO 96/34949), the Bet v 1 promoter (Swoboda, I., et al., Plant, Cell and Env. 18, 865-874, 1995), the Vst1 promoter (Fischer, R., Dissertation, Univ. of Hohenheim, 1994; Schubert, R., et al. Plant Mol. Biol. 34, 417-426, 1997), the sesquiterpene cyclase promoter (Yin, S., et al., Plant Physiol. 115, 437-451, 1997) and the gstA1 promoter (Mauch, F. and Dudler, R., Plant Physiol. 102, 1193-1201, 1993), MS59 (WO 99/50428), ICS (WO 99/50423) or any other pathogen-inducible promoter. If more than one anti-fungal agent is to be expressed, the same or different regulatory regions may be used for different anti-fungal agents. The DNA construct may then be transformed into a plant.

Transformation in a large number of plant species (both Dicotyledoneae and Monocotyledoneae) is now achievable. In principle, any transformation method may be used to introduce chimaeic DNA into a suitable ancestor cell, as long as the cells are capable of being regenerated into whole plants. Examples of suitable transformation methods include the calcium/polyethylene glycol method for protoplasts (Krens, F.A. et al., 1982, Nature 296, 72-74; Negrutiu I. et al, June 1987, Plant Mol. Biol. 8, 363-373); electroporation of protoplasts (Shillito R.D. et al., 1985 Bio/Technol. 3, 1099-1102); microinjection into plant material (Crossway A. et al., 1986, Mol. Gen. Genet. 202, 179-185); DNA or RNA-coated particle bombardment of various plant material (Klein T.M. et al., 1987, Nature 327, 70); infection with (non-integrative) viruses and the like. A preferred method according to the invention comprises Agrobacterium-mediated DNA transfer. Especially preferred is the so-called binary vector technology as disclosed in EP A 120 516 and U.S. Pat. No. 4,940,838.

Following transformation, plant cells or cell groupings are typically selected for the presence of one or more markers encoded by plant expressible genes co-transferred with the nucleic acid sequence to be introduced. This is followed by regeneration into a whole plant of the transformed material.

Although considered somewhat more recalcitrant towards genetic transformation, monocotyledonous plants are amenable to transformation and fertile transgenic plants can be regenerated from transformed cells or embryos, or other plant material. At present, preferred methods for transformation of monocots include microprojectile bombardment of embryos, explants or suspension cells, and direct DNA uptake or electroporation (Shimamoto, et al, 1989, Nature 338, 274-276). Transgenic maize plants have been obtained by introducing the Streptomyces hygroscopicus bar-gene, which encodes phosphinothricin acetyltransferase (an enzyme which inactivates the herbicide phosphinothricin), into embryogenic cells of a maize suspension culture by microprojectile bombardment (Gordon-Kamm, 1990, Plant Cell, 2, 603-618). The introduction of genetic material into aleurone protoplasts of other monocot crops, such as wheat and barley has been reported (Lee, 1989, Plant Mol. Biol. 13, 21-30). Wheat plants have been regenerated from embryogenic suspension culture by selecting only the aged compact and nodular embryogenic callus tissues for the establishment of the embryogenic suspension cultures (Vasil, 1990 Bio/Technol. 8, 429-434). The combination with transformation systems for these crops enables the application of the present invention to monocots. Monocotyledonous plants, including commercially important crops such as rice and corn are also amenable to DNA transfer by Agrobacterium strains (vide WO 94/00977; EP 0 159 418 B1; Gould J, Michael D, Hasegawa O, Ulian E.C., Peterson G, Smith R.H., (1991) Plant. Physiol. 95, 426-434). More recently, successful transformation of monocotyledonous plants, especially cereals, has been reported. For example, transformation of rice has been described in WO 94/00977, U.S. Pat. No. 5,591,616 and EP 0 672 752; transformation of wheat has been described in U.S. Pat. No. 5,631,152, WO 97/48814; transformation of sorghum has been described in WO 98/49332; transformation of barley and wheat has been described in WO 98/48613; transformation of maize has been described in WO98/32326; transformation of banana has been described in U.S. Pat. No. 5,792,935; and transformation of barley has been described in WO 99/04618.

Following DNA transfer and regeneration, putatively transformed plants may be evaluated (for instance using Southern analysis) for the presence of the chimeric DNA, determination of copy number and/or genomic organization. After the optional initial analysis step, transformed plants showing the desired copy number and expression level of the newly introduced chimeric DNA may be tested for resistance levels against a pathogen. Other evaluations may include the testing of pathogen resistance under field conditions, checking of fertility, yield, and other characteristics. Such testing is now routinely performed by persons having ordinary skill in the art. Following evaluation, the transformed plants may be grown directly or used as parental lines in the breeding of new varieties or in the creation of hybrids or the like.

Advantageously, according to the present invention, the amount of anti-fungal compound/fungicide applied (in a single application) to a plant, genetically modified to introduce or improve plant resistance to fungi, may be reduced from an ordinary amount (which is the amount used by a farmer to control disease) which is typically 0.1 g to 12 kg ai (active ingredient) per hectare, preferably from 1 g to 6 kg ai per hectare, more preferably from 1 g to 2 kg ai per hectare) to an amount which is about 50% lower than the ordinary amount (i.e., 0.05g to 6 kg ai per hectare). For example, chlorothalonil, which may normally be applied on a specific crop at a rate of 1.3 kg ai per hectare, may be used at a rate of 0.65 kg ai per hectare. Further preferably, the amount of anti-fungal compound/fungicide applied to a plant (which is genetically modified to introduce or improve plant resistance to fungi) may be reduced by about 75% of the ordinary amount (i.e., 0.025 g to 3 kg ai per hectare).

Advantageously, according to the present invention, the frequency and/or rate of application of an anti-fungal compound/fungicide to a plant (which is genetically modified to introduce or improve resistance to fungi) may be reduced compared to frequency and/or rate of application to non-modified crops. The frequency of application of an anti-fungal compound/fungicide to a plant may reduced from, for example, an average application rate of about once every 10 days to an average application rate of about once every 15 to 20 days.

The method according to the present invention confers several economic and environmental advantages. The synergistic effect demonstrated enables the anti-fungal compounds/fungicides to be used at a reduced rate, thereby lowering material and labour costs, minimising adverse effects on the environment and prolonging the shelf-life of products, such as fruit and seed/grain.

Advantageously, the method according to the present invention enables an increase in plant resistance to fungi without adversely affecting the yield characteristics of a plant. An increase in overall yield may be seen due to reduction or elimination of loss resulting from fungal damage.

Depending on the strength and type of the promoter used, it can take some time for a genetically engineered plant comprising the recombinant DNA to complete the HR response. Therefore, application of an anti-fungal compound/fungicide will help combat the spread and attack of fungal pathogens in the period before the fungicide takes effect. Once the genetic anti-fungal effect takes place, a synergistic interaction between the fungicide expressed in the plant and the anti-fungal compound/fungicide may be demonstrated.

Further advantageously, the method according to the present invention may be used as a preventative measure or as a means to counteract further fungal attack and/or to inhibit or decrease the rate of spread of fungi through and/or on the plant.

The method according to the present invention is also suitable for use on plants which have been further genetically modified to introduce alternative or further traits, such as herbicide resistance, insect/acarid resistance, a trait resulting in modified oil or starch content or any other trait.

The present invention will now be further described with reference to the following Figures:

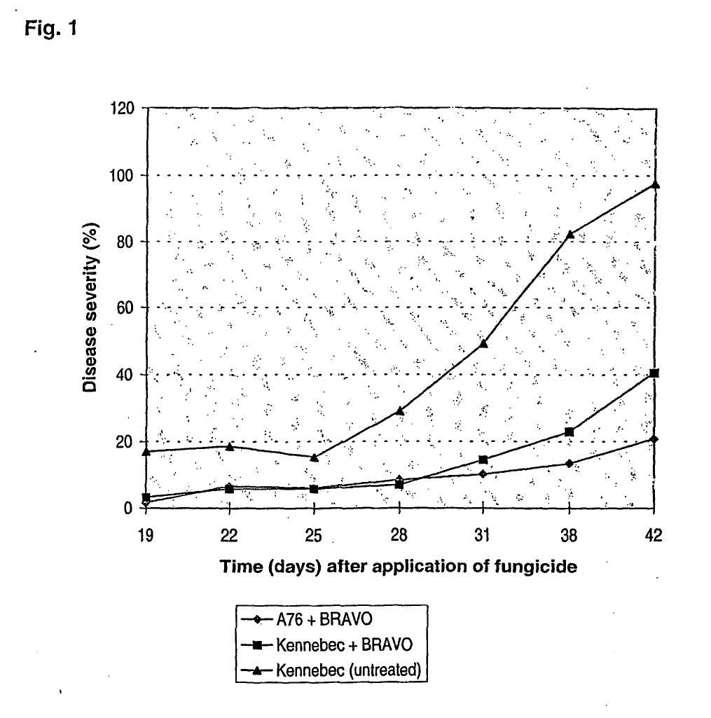

FIG. 1

The severity of late blight on transgenic Russet Burbank and Kennebec potato varieties is shown. The level of disease in Russet Burbank (comprising an avilulence gene and a corresponding resistance gene) treated with anti-fungal compound chlorothalonil (Bravo Weather Stik™) compared with levels of disease in Kennebec (a variety more resistant to Phlytophthlora infection than variety Russet Burbank) also treated with chlorothalonil is shown. Untreated Kennebec was used as a control. Levels of disease over a 42 day time course are shown.

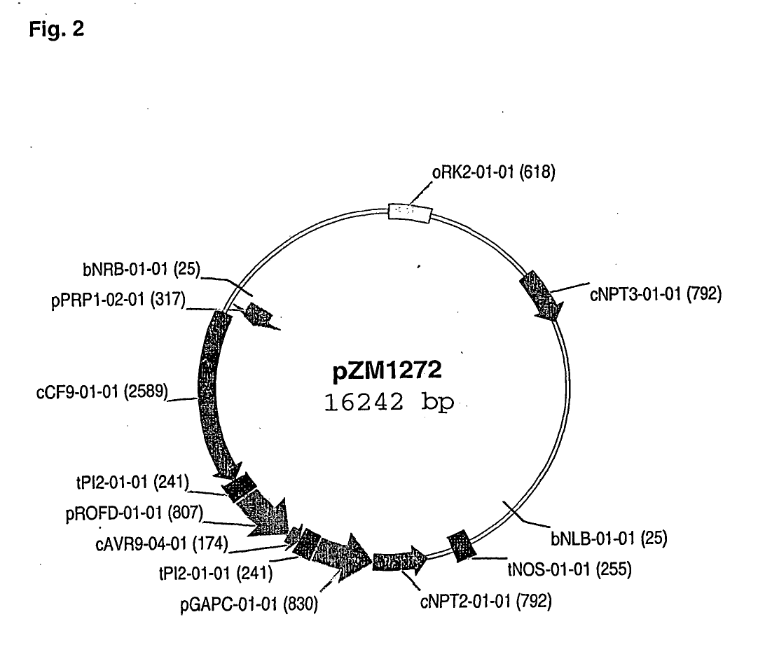

FIG. 2

A diagrammatic representation of the construct introduced to potato variety Russet Burbank in the example given below is shown.

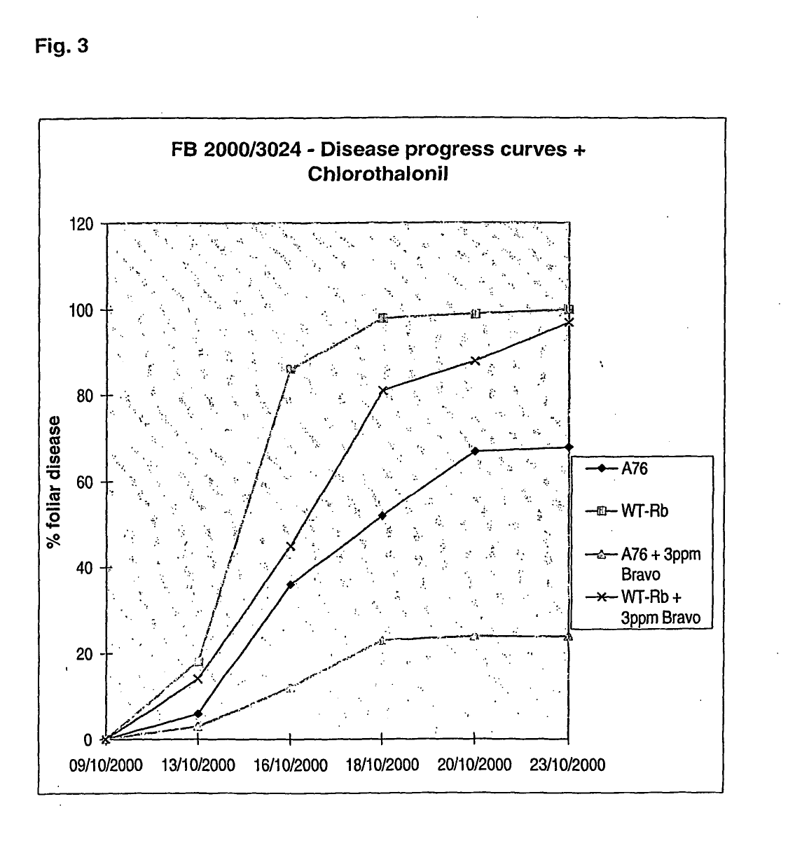

FIG. 3

Disease progress curve for Phytophthora infestans and chlorothalonil (BRAVO 720™) is shown. Wt−Rb=wildtype Russet Burbank potato and A76=genetically modified Russet Burbank (for description see Example 2).

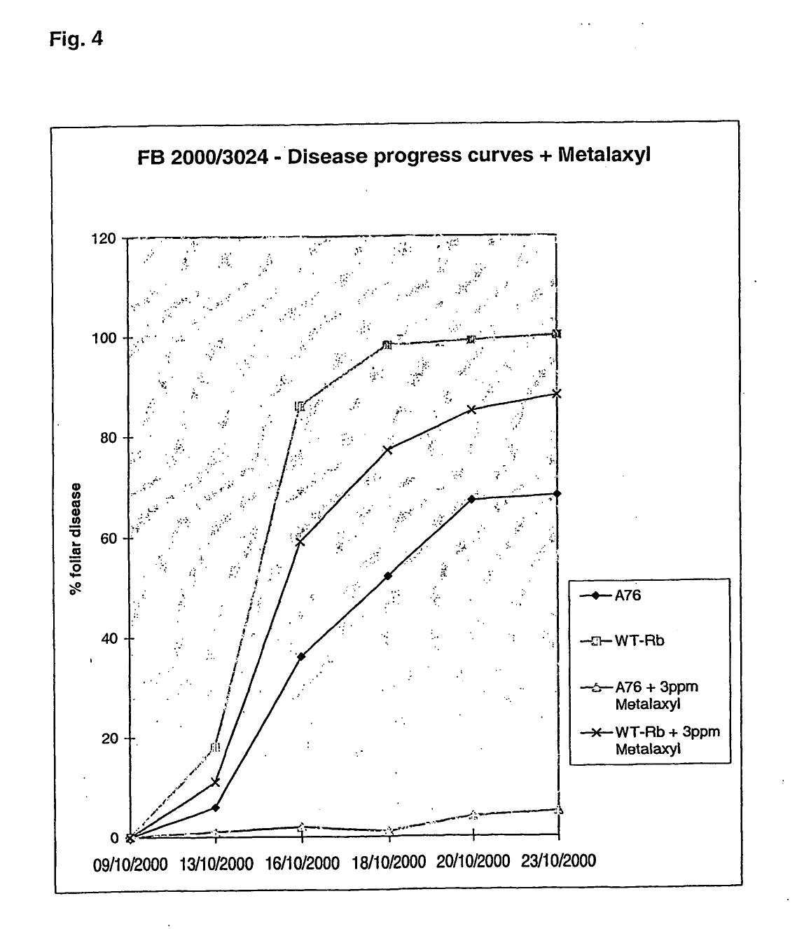

FIG. 4

Disease progress curve for Phytophthora infestans and metalaxyl is shown. Wt−Rb=wildtype Russet Burbank potato and A76=genetically modified Russet Burbank (for description see Example 2).

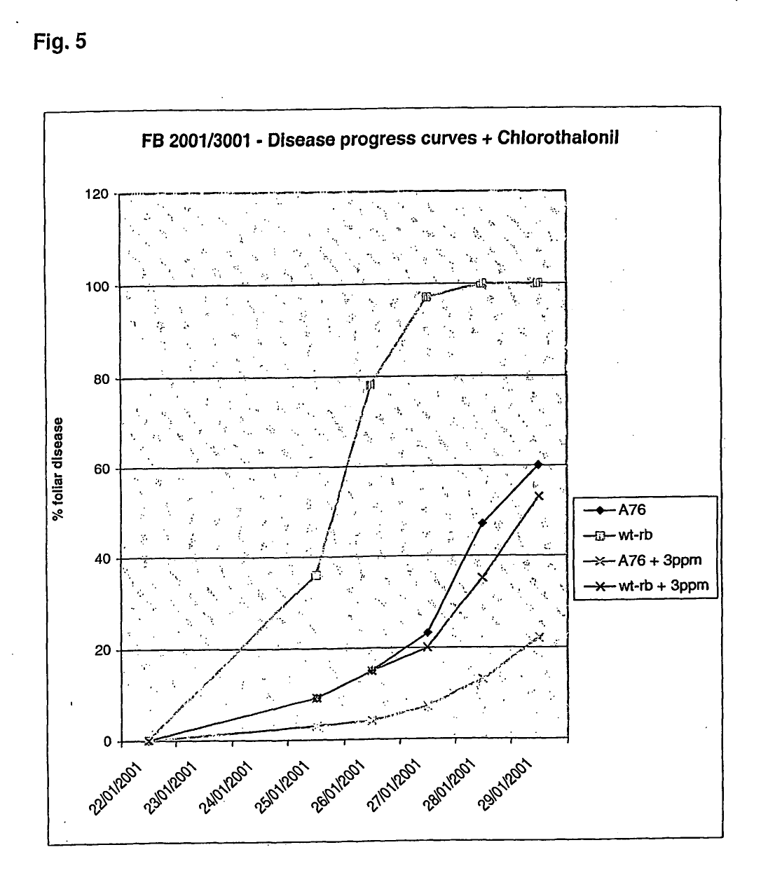

FIG. 5

Disease progress curve for Phytophthora infestans and chlorothalonil (BRAVO 72™) is shown. Wt−Rb=wildtype Russet Burbank potato and A76=genetically modified Russet Burbank (for description see Example 2).

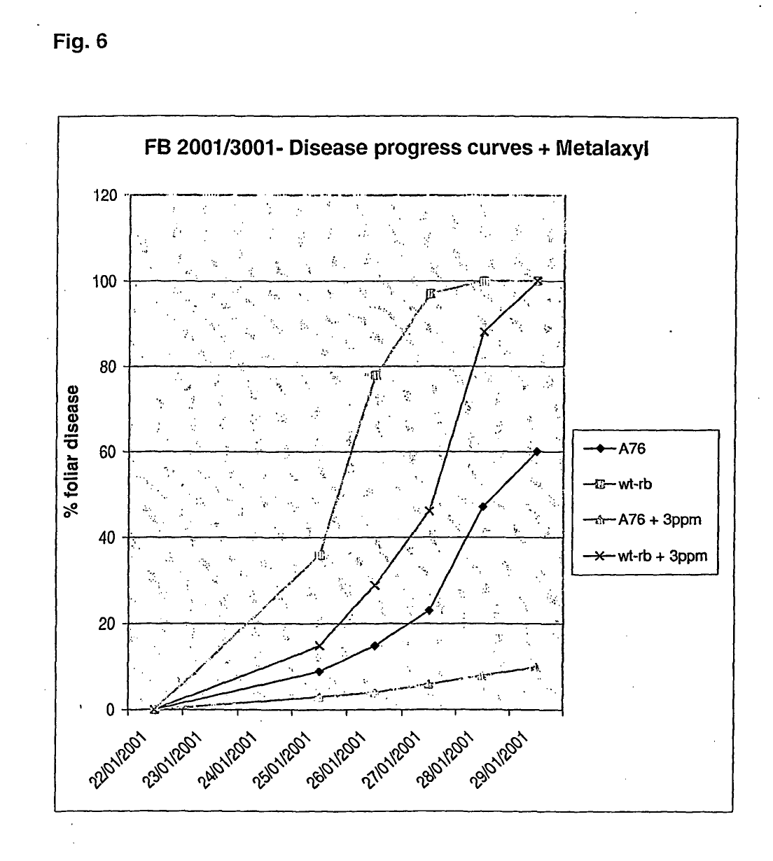

FIG. 6

Disease progress curve for Phytophthora infestans and metalaxyl is shown. Wt−Rb=wildtype Russet Burbank potato and A76=genetically modified Russet Burbank (for description see Example 2).

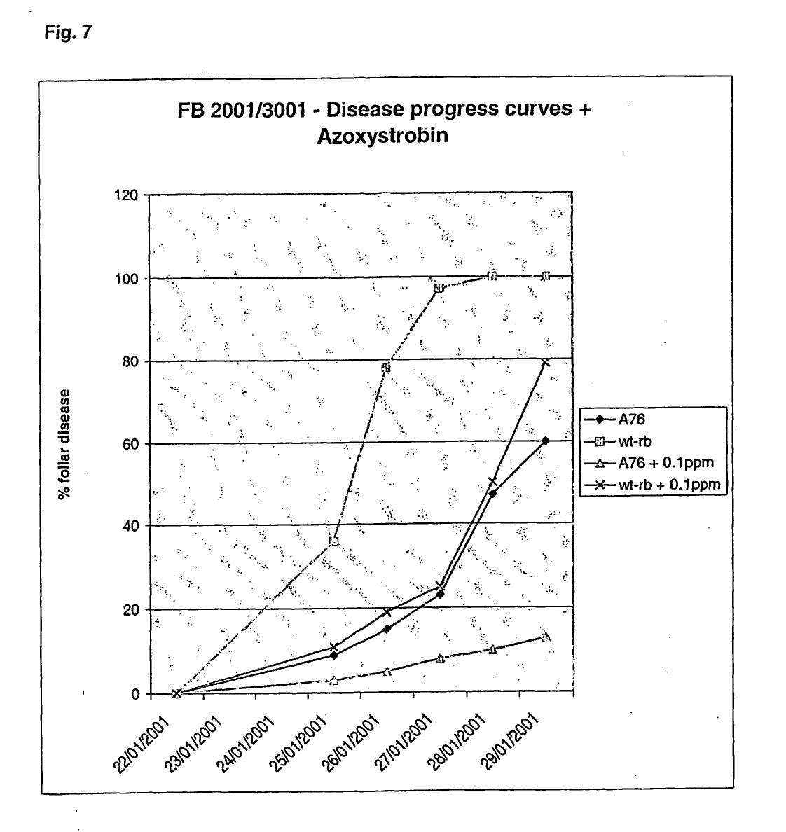

FIG. 7

Disease progress curve for Phytophthora infestans and azoxystrobin is shown. Wt−Rb=wildtype Russet Burbank potato and A76=genetically modified Russet Burbank (for description see Example 2).

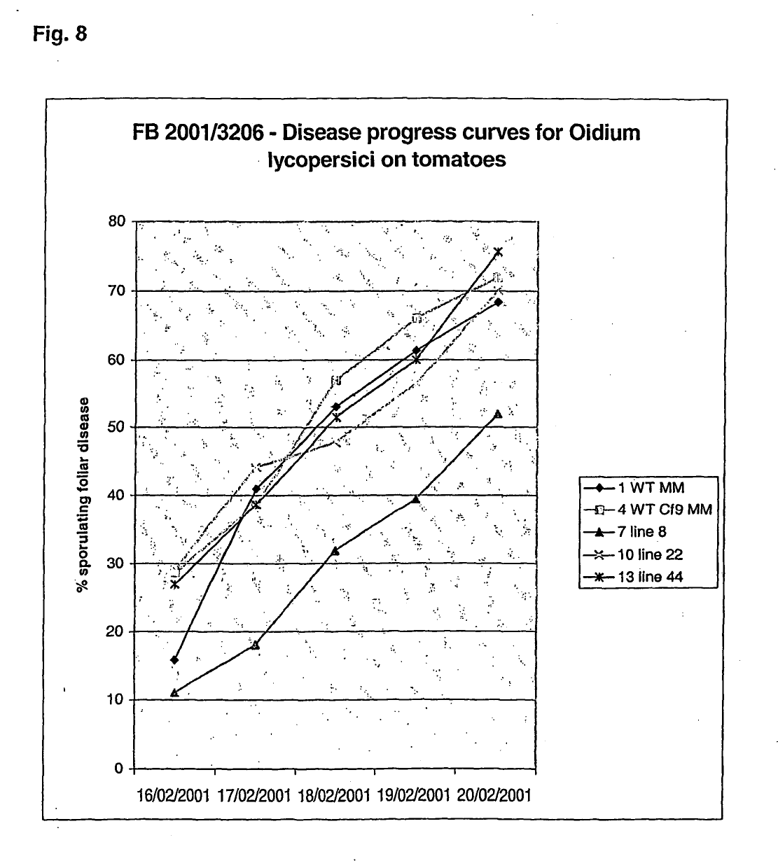

FIG. 8

Disease progress curve for Oiduim lycopersici on wild-type and transgenic tomatoes without application of fungicide is shown.

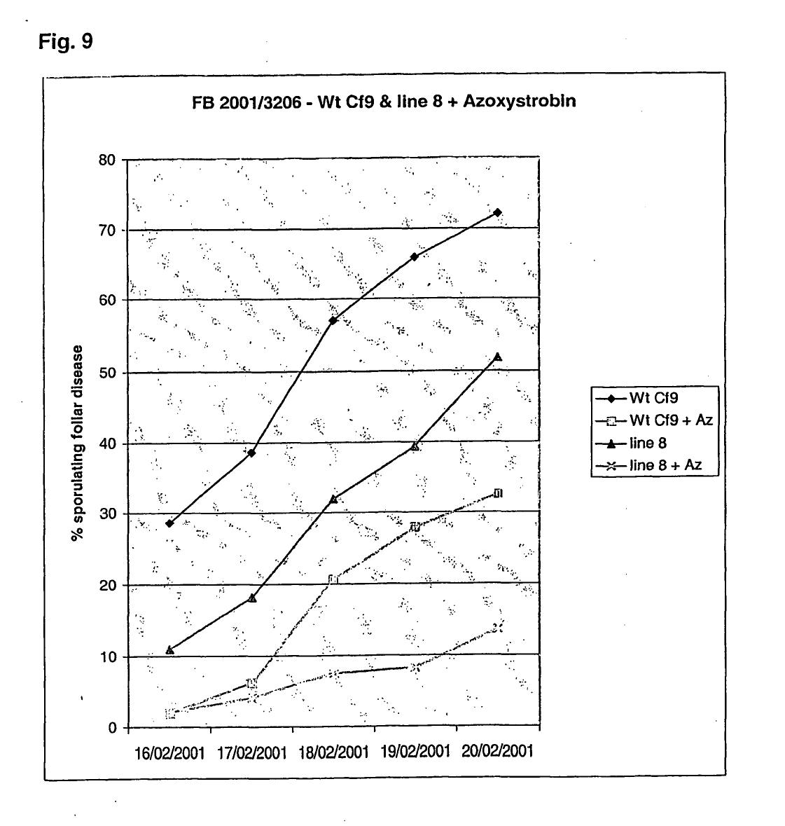

FIG. 9

Disease progress curve for wild-type Cf9 and transgenic line 8 tomatoes upon application of azoxystrobin is shown.

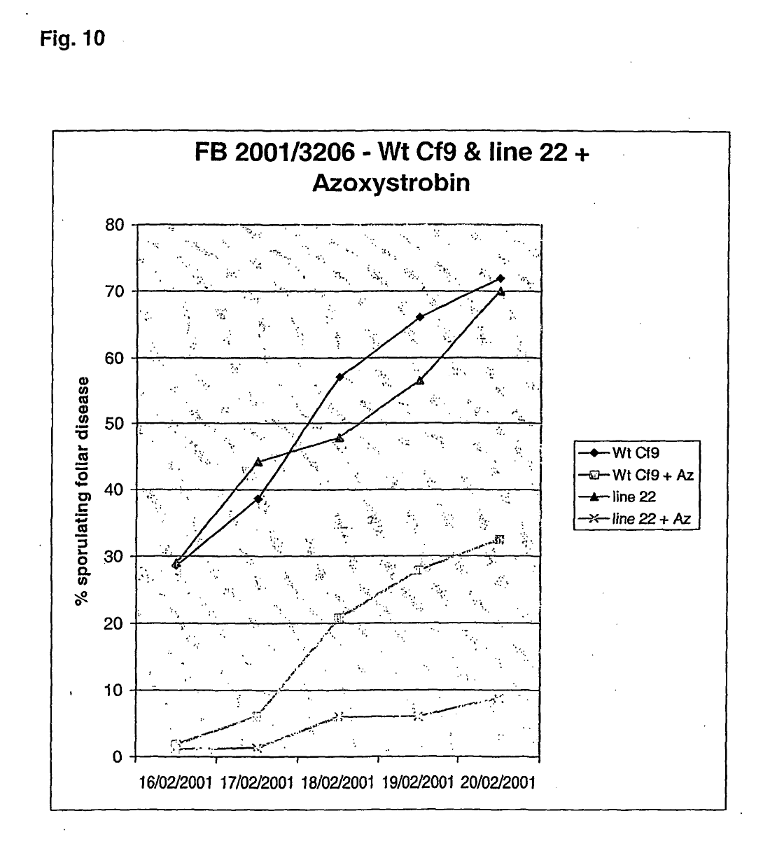

FIG. 10

Disease progress curve for wild-type Cf9 and transgenic line 22 tomatoes upon application of azoxystrobin is shown.

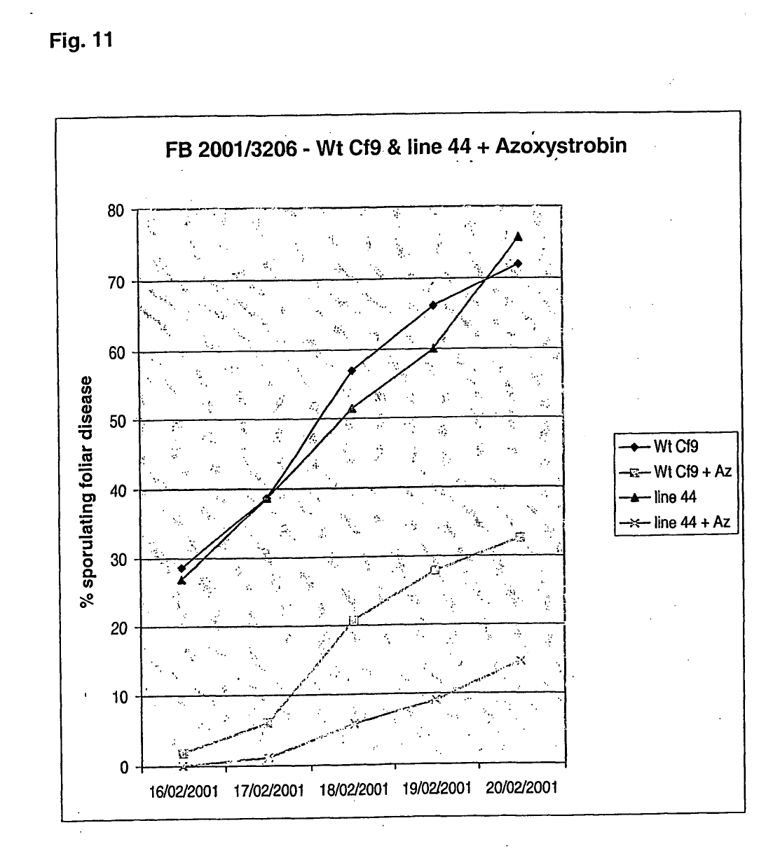

FIG. 11

Disease progress curve for wild-type Cf9 and transgenic line 44 tomatoes upon application of azoxystrobin is shown.

The present invention will now be further defined with reference to the following examples.

### EXAMPLES

Standard methods for isolation, manipulation and amplification of DNA, as well as suitable vectors for replication of recombinant DNA, suitable bacterium strains, selection markers, media and the like are described, for instance in Maniatis et al., molecular cloning: A Laboratory Manual 2nd. edition (1989) Cold Spring Harbor Laboratory Press; DNA Cloning: Volumes I and II (D.N. Glover ed. 1985); and in: From Genes To Clones (E.-L. Winnacker ed. 1987).

### Example 1

Method

Experiments were carried out to determine the effects of applying a reduced concentration of fungicide to potato plants, which had been genetically modified to introduce or improve resistance to fungi, as compared to control plants. Potato transformation is preferably done essentially as described by Hoekema et al. (Hoekema, A. et al., Bio/Technology 7, 273-278, 1989).

An avirulence gene, avr9 from Cladosporium fulvum, together with resistance gene Cf9 from tomato, was introduced into potato variety Russet Burbank using conventional Agrobacterium. transformation. The construct used was prp1-Cƒ9+ƒdrolD-Avr9 (as shown in FIG. 2). One of the lines, namely line A76, performed well in a field trial and was subsequently selected for use in further field trials. Fungal pathogen Phytophthlora infestans was sprayed onto plots containing potato variety Russet Burbank (a variety susceptible to Phytophthora infection) and potato variety Kennebec (a variety which is more resistant to Phytophthora infection than variety Russet Burbank). Commercially available fungicide, Bravo Weather Stik™ (chlorothalonil) was diluted to 1/4 of the strength normally used on potato plants, 1/4 strength being 325 g ai per hectare (undiluted application rate typically being 1.3 kg ai per hectare). The diluted fungicide was then applied to potato variety Russet Burbank, comprising the construct, and to the non-transformed variety Kennebec. The fungicide was first applied to the plants 6 to 8 weeks after infection and was applied every 7 to 10 days thereafter at a 1/4 dilution. A control experiment omitting the fungicide was also set up. The level of disease was monitored over a 42 day time period (with assessment at 19, 22, 25, 28, 31, 38 and 42 days after the first application of fungicide).

Results

The results obtained are shown in FIG. 1 and Table 1 below. In the case of the genetically modified variety, continued resistance to disease was shown over the 42 day time period. The results shown FIG. 1 demonstrate that fungicide applied at a reduced concentration to susceptible variety (Russet Burbank (modified to introduce an avirulence gene)) provides a level of resistance which is even better than the level of resistance obtained following application of fungicide (at a reduced concentration) to the more resistant variety, Kennebec.

TABLE 1

| DAYS AFTER FIRST | A76 + | KENNEBEC + | |
|---|---|---|---|
| FUNGICIDE | ¼ | ¼ | KENNEBEC |
| APPLICATION | BRAVO | BRAVO | UNTREATED |
| 19 | 1.69 | 3.50 | 17.13 |
| 22 | 6.81 | 6.06 | 18.75 |
| 25 | 6.25 | 5.94 | 15.44 |
| 28 | 9.06 | 7.19 | 28.88 |
| 31 | 10.88 | 15.13 | 49.25 |
| 38 | 14.13 | 23.25 | 81.88 |
| 42 | 21.56 | 40.63 | 97.13 |

### Example 2

Method

Potato plants 5 to 6 weeks of age (originating from tissue culture plantlets) were removed from tissue culture medium, cultivated in a peat-based compost and used in the evaluation. The parental Russet Burbank germplasm (Wt-St/Rb-1) was compared with the transformed 1272-St/Rb-1/76 (A76). The plants were grown in constant environment growth rooms with a 21° C. day temperature and 16-18° C. at night. Day length was 16 hours (light intensity 100 μM photons/m⁻² .s) and relative humidity was 60% during day and 95% at night. The chemicals were applied by foliar spray. Application rates were selected as those where fungal control was breaking down on the parental germplasm. Chemicals used were chlorathalonil (BRAVO 720™, 54%w/w, 720 g/l SC), metalaxyl (240 g/l EC) and azoxystrobin (250 g/l SC). Plants were inoculated with a suspension of Phlytopthora infestans sporangia (a US1 isolate, which is maintained on detached tomato foliage) by foliar spray four days 5000 sporangia/ml) after chemical application.

Whilst the inoculum was still wet on the foliage, the plants were placed in a dew chamber (21° C., 100% relative humidity) for approximately 20 hours. After the inoculation the plants were replaced in the growth room under the conditions as described above.

Results

The results obtained are shown in Tables 2 and 3 and FIGS. 3 to 7. Tables 2 and 3 provide a comparison of the observed disease control compared to the expected disease control. The values were derived by using Colby's formula, as shown below.

E=(100−% disease control for untreated genetically modified plant alone)×(% disease control for relevant chemical & rate alone)/100+% disease control for untreated genetically modified plant alone

E=Expected% disease control

The observed values were calculated as percentage disease control relative to Wt-St/Rb-1 (wild-type). The expected values were calculated using Colby's formula and were based on an assumption of independent action between the line effect and the chemical effect. The results shown in Tables 1 and 2 (2 different experiments) show that, for all rates of metalaxyl and at all assessment points, the performance of metalaxyl in combination with A76 is better than expected and therefore indicative of synergy. A76 in combination with chlorothalonil and A76 in combination with azoxystrobin also demonstrate better than expected performance, which again is indicative of synergy. The synergistic effect seen is also clearly illustrated in FIGS. 3 to 7.

TABLE 2
FB 2000/3024 Comparison of Observed and Expected Responses

| | Oct. 13, 2000 | | | Oct. 16, 2000 | | | Oct. 18, 2000 | | | Oct. 20, 2000 | | |
|---|---|---|---|---|---|---|---|---|---|---|---|---|
| | O | E | O-E | O | E | O-E | O | E | O-E | O | E | O-E |
| A76 + Chlorothalonil 10 ppm | 88.73 | 92.49 | −3.76 | 95.36 | 94.65 | 0.71 | 87.77 | 91.33 | −3.56 | 79.74 | 84.98 | −5.24 |
| A76 + Chlorothalonil 3 ppm | 83.1 | 73.73 | 9.37 | 86.09 | 78.12 | 7.97 | 76.41 | 56.09 | 20.32 | 75.7 | 39.95 | 35.75 |
| A76 + Metalaxyl 30 ppm | 100 | 96.25 | 3.75 | 100 | 97.08 | 2.92 | 97.95 | 94.58 | 3.37 | 97.97 | 89.77 | 8.2 |
| A76 + MetaIaxyl 10 ppm | 94.37 | 88.74 | 5.63 | 96.6 | 87.84 | 8.76 | 97.95 | 75.07 | 22.88 | 96.96 | 48.83 | 48.13 |
| A76 + Metalaxyl 3 ppm | 94.37 | 79.35 | 15.02 | 97.68 | 71.31 | 26.37 | 97.95 | 58.26 | 39.69 | 95.95 | 42.01 | 53.94 |

TABLE 3
FB 2001/3001 Comparison of Observed and Expected Responses

| | Jan. 25, 2001 | | | Jan. 26, 2001 | | | Jan. 27, 2001 | | | Jan. 28, 2001 | | | Jan. 29, 2001 | | |
|---|---|---|---|---|---|---|---|---|---|---|---|---|---|---|---|
| | O | E | O-E | O | E | O-E | O | E | O-E | O | E | O-E | O | E | O-E |
| A76 + Chlorothalonil 10 ppm | 97.2 | 97.0 | 0.2 | 97.4 | 98.2 | −0.8 | 97.9 | 97.8 | 0.1 | 99.0 | 93.8 | 5.2 | 97.0 | 90.1 | 6.9 |
| A76 + Chlorothalonil 3 ppm | 91.7 | 98.3 | −1.6 | 94.9 | 96.2 | −1.3 | 92.7 | 94.5 | −1.7 | 86.9 | 83.2 | 3.6 | 77.9 | 67.2 | 10.7 |
| A76 + Metalaxyl 10 ppm | 97.2 | 98.3 | 3.9 | 98.7 | 96.2 | 2.5 | 99.0 | 94.2 | 4.8 | 98.0 | 80.4 | 17.6 | 97.0 | 55.4 | 41.6 |
| A76 + Metalaxyl 3 ppm | 91.7 | 88.8 | 2.8 | 94.9 | 92.6 | 2.3 | 93.8 | 87.2 | 6.5 | 91.9 | 57.9 | 34.1 | 89.9 | 38.1 | 51.8 |
| A76 + Azoxystrobin 0.3 ppm | 97.2 | 95.5 | 1.7 | 98.7 | 97.2 | 1.5 | 97.9 | 95.6 | 2.4 | 98.0 | 87.1 | 10.9 | 97.0 | 74.6 | 22.4 |
| A76 + Azoxystrobin 0.1 ppm | 91.7 | 91.8 | −0.1 | 98.6 | 95.1 | −1.6 | 91.7 | 93.1 | −1.4 | 89.9 | 76.1 | 13.9 | 86.9 | 51.1 | 35.8 |
| A76 + Azoxystrobin 0.03 ppm | 86.1 | 85.1 | 1.0 | 91.0 | 91.0 | 0.0 | 89.6 | 84.2 | 5.4 | 84.9 | 56.4 | 28.4 | 63.8 | 38.7 | 25.1 |

0 = Observed % disease control
E = Expected % disease control

### Example 3

Method

Tomato plants (variety Moneymaker), already endogenously expressing the Cf9 protein were transformed with constructs harbouring the avr9 gene under control of the prp1 pathogen inducible promoter. Two constructs were used, namely prp1::35S::omega::Pr1a-Avr9-Tpi (pMOG980) and prp1::35S.:omega::Pr1a -Avr9(R8K)-Tpi (pMOG981). The two constructs used were identical except for a point mutation in pMOG981 resulting in replacement of the Arg residue at position 8 for a Lys residue in the mature Avr9 protein. The promoter used was a chimeric promoter consisting of the prp1 regulatory region (as described by Martini 1993, Molecular General Genetics 236: 179-186), the 35S minimal promoter (Guilley et al. 1982, Cell 30: 763-773) and the 5′TMV-U1 (omega) leader (Gallie et al. 1987, Nucleic Acids Research 15: 8693-8709). The sequence encoding the PR1a signal peptide (Pfitzner et al. 1988, Molecular General Genetics 211:290-295) was cloned in frame with the DNA sequence encoding the mature Avr9 protein (Van Kan et al., Molecular Plant-Microbe Interactions 4: 52-59). The terminator from the protease inhibitor protein gene from potato (Thornburg et al. 1987, Proc. Natl. Acad. Sci. USA 84: 744-748) was used in both prp1-Avr9 cassettes.

Transformations were done essentially according to the method described in Van Roekel et al. (Plant Cell Rep. 12, 644-647, 1993). Transformation with the non-mutated avr9 gene resulted in tomato line 8, while transformation with the mutated avr9 gene gave lines 22 and 44 as result. The transgenic tomato plants were grown and seeds were obtained. S1 plants from the three lines were originated from these seeds and were grown in a soil based potting compost under the same conditions as the potato plants described in Example 2. Fungicide used for application was azoxystrobin (250 g/l SC).

For the powdery mildew (Oidium lycopersici) assay, leaves were excised 24 hours post-chemical application. Detached fully expanded terminal leaflets had their petioles inserted into 0.8% (w/v) tap water agar in a 25 cm×25 cm bioassay plate. The leaflets were inoculated by tapping sporulating tomato leaflets infested with Oidium above them, allowing spores to drop onto the leaf surface. Lids were replaced on the dishes and the dishes were placed in a constant environment room (21.5° C., day length 16 hours) under a light bank (4760 lux).

Results

The results are shown in FIGS. 8 to 11 and Table 4.

Table 4 below compares the observed disease control with the expected disease control for three tomato transgenics. Observed values in the tables are calculated as percentage disease control relative to Wt-Le/MM/Cf9. Expected values are generated using Colby's Formula and are based on an assumption of independent action between the line effect and the chemical effect. From the table it can be seen that for all three lines, the performance of Azoxystrobin in combination with the transgenic line is better than expected, indicative of synergy.

TABLE 4

| | Feb. 16, 2001 | | | Feb. 17, 2001 | | | Feb. 18, 2001 | | | Feb. 19, 2001 | | | Feb. 20, 2001 | | |
|---|---|---|---|---|---|---|---|---|---|---|---|---|---|---|---|
| | O | E | O-E | O | E | O-E | O | E | O-E | O | E | O-E | O | E | O-E |
| 980 Le/MM/Cf9-8 + Az 0.1 ppm | 93.01 | 97.5 | −4.49 | 89.64 | 92.53 | −2.89 | 86.84 | 79.68 | 7.16 | 87.5 | 74.81 | 12.69 | 81.11 | 67.47 | 13.64 |
| 981 Le/MM/Cf9-22 + Az 0.1 ppm | 96.01 | 93.41 | 2.6 | 96.55 | 81.74 | 14.81 | 89.47 | 69.49 | 19.98 | 90.91 | 63.9 | 27.01 | 87.84 | 56.11 | 31.73 |
| 980 Le/MM/Cf9-44 + Az 0.1 ppm | 100 | 93.9 | 6.1 | 97.12 | 84.21 | 12.91 | 89.72 | 67.22 | 22.5 | 86.36 | 61.62 | 24.74 | 79.57 | 52.54 | 27.03 |

1. A method for introducing and/or improving plant resistance to attack and/or spread of fungal pathogens, said method comprising applying at least one anti-fungal compound to a plant or plant part genetically modified to express at least one agent able to trigger a hypersensitive response in a plant, wherein said anti-fungal compound synergistically enhances said plant resistance.

2. A method according to claim 1, wherein said agent able to trigger a hypersensitive response in a plant is a pathogen avirulence gene encoding a specific elicitor, or a functional part thereof which acts in combination with a corresponding resistance gene, wherein said corresponding resistance gene is either already present in said plant or plant part and/or wherein said corresponding resistance gene is introduced into said plant or plant part.

3. A method according to claim 2, wherein said avirulence gene is Avr9 from Cladosprium fulvum and said corresponding resistance gene is Cf9 from tomato.

4. A method according to any of claims 1 to 3, wherein said anti-fungal compound is selected from phenylamides, strobilurins or chlorothalonil or a derivative thereof.

5. A method according to claim 4, wherein said phenylamide is metalayxyl and said strobilurin is azoxystrobin.

6. A method according to claim 1, wherein a combination of more than one anti-fungal compounds is used, optionally together with other agricultural chemical compounds.

7. A method according to any of claims 1 to 6, wherein the amount of antifungal compound applied to said genetically modified plant is reduced from an ordinary amount to an amount which is about 50% lower than the ordinary amount.

8. A method according to any of claims 1 to 6, the amount of antifungal compound applied to said genetically modified plant is reduced from an ordinary amount to an amount which is about 75% lower than the ordinary amount.

9. A method according to any of claims 1 to 8, wherein the frequency and/or rate of application of said anti-fungal compound to said genetically modified plant is reduced from an average application rate of about once every 10 days to an average application rate of about once every 15 to 20 days.

10. A method according to any of claims 1 to 9, wherein said plant or plant part is selected from potato, tomato, banana, sugar beet, tobacco, maize, rice or wheat.

11. A method according to claim 10, wherein said plant is potato or tomato.

12. A method according to claim 10 or 11, wherein said plant is potato.

13. A method according to any of claims 10 to 12, wherein said plant is a potato plant transformed with an Avr9 gene and a Cf9 gene and wherein said anti-fungal compound is chlorothalonil.

14. A method according to any of claims 10 to 12, wherein said plant is a potato plant transformed with Avr9 gene and a Cf9 gene and wherein said anti-fungal compound is metalaxyl.

15. A method according to any of claims 10 to 12, wherein said plant is a potato plant transformed with an Avr9 gene and a Cf9 gene and wherein said anti-fungal compound is azoxystrobin.

16. A method according to any of claims 10 to 12, wherein said plant is a tomato plant transformed with an Avr9 gene and a Cf9 gene and wherein said anti-fungal compound is azoxystrobin.

17. A method according to any of claims 1 to 16, wherein said fungal pathogen is selected from Magnaporthe grisea, Erysiphe graminis, Septoria tritici, Botrytis cinerea, Cladosporium spp., Oidium lycopernicon, Phoma spp., Phytophthora infestans, Sclerotinia spp., Peronospora tabacina, Stagonospora nodorum, and Mycosphaerella spp.

18. A plant or plant part having improved resistance to attack and spread of fungal pathogens obtained by a method according to any of claims 1 to 17.

19. A plant or plant part according to claim 18 having been treated with a curative anti-fungal compound.

20. A plant or plant part having been genetically modified to introduce a pathogen avirulence gene encoding a specific elicitor or a functional part thereof, wherein said plant either already comprises a corresponding resistance gene or wherein said corresponding resistance gene is introduced into said plant or plant part and wherein said plant has been treated with at least one anti-fungal compound.
